(19) United States
(12) Patent Application Publication
(10) Pub. No.: US 2022/0071705 A1
(43) Pub. Date: Mar. 10, 2022

(54) ULTRASOUND-ENHANCED LASER THROMBOLYSIS WITH ENDOVASCULAR LASER AND HIGH-INTENSITY FOCUSED ULTRASOUND

(71) Applicant: University of Kansas, Lawrence, KS (US)

(72) Inventors: Xinmai Yang, Lawrence, KS (US); M Laird Forrest, Lawrence, KS (US)

(21) Appl. No.: 17/466,780

(22) Filed: Sep. 3, 2021

Related U.S. Application Data
(60) Provisional application No. 63/074,798, filed on Sep. 4, 2020.

(51) Int. Cl. A61B 18/24; A61F 2/01

(52) U.S. Cl. CPC A61B 18/245; A61B 2018/00994; A61F 2/013

(57) ABSTRACT

A system for thrombolysis includes an optical energy source, an ultrasound transducer, and an optical conduit for insertion into a vessel. The optical conduit directs optical energy from the optical energy source to a target location at a terminal end of the optical conduit.

### PRIORITY

This application claims priority to and benefit of United States Provisional Patent Application No. 63/074,798 titled “ULTRASOUND-ENHANCED LASER THROMBOLYSIS WITH ENDOVASCULAR LASER AND HIGH-INTENSITY FOCUSED ULTRASOUND” filed Sep. 4, 2020, the entirety of which is incorporated herein by reference.

### STATEMENT OF GOVERNMENT INTEREST

This invention was made with government support under Grant No. HL147783 awarded by the National Institutes of Health. The government has certain rights in the invention.

### BACKGROUND OF THE DISCLOSURE

Deep vein thrombosis (DVT), characterized by excessive blood clot (thrombus) formation in veins, is a major disease affecting more than 10 million people worldwide each year. Medical complications associated with DVT include pulmonary embolism (PE) and postthrombotic syndrome (PTS). PE is an acute life-threatening complication and is known to be induced by the debris of the blood clots when they break off from the central clot and block smaller blood vessel flowing into the lungs. The blockage of blood supply can cause severe damages to the lungs, resulting in breathing difficulties, and ultimately leading to death. Annually, as many as 100,000 patients die from PE in the United States. PTS is another costly chronic condition that develops in 30% to 75% of patients with DVT. PTS includes redness, swelling, ulcers and chronic leg pain, and it can lead to life-long suffering and potentially disability. The annual costs for DVT-related complications are $7 to $10 billion in the United States. Worldwide, the total cost can be as high as $69 billion annually.

Among the current standards of care, anticoagulants can prevent thrombus propagation; however, they do not dissolve existing thrombi and re-canalize vessels. Thrombolytic therapy that has been historically performed to dissolve clots may greatly increase the risk of bleeding, and their introduction may require hospitalization. Ultrasound-based treatment techniques have been evaluated as methods to induce effective thrombolysis. The advantage of ultrasound-based techniques is that they can dissolve blood clots quickly and re-canalize vessels noninvasively through cavitation. While ultrasound-based techniques may quickly remove blood clots noninvasively, these techniques require high acoustic peak negative pressure (as high as 19 MPa) at relatively low ultrasound frequencies such as 500 kHz or 1 MHz. In order to achieve high ultrasound pressure and deliver treatment to a blood clot, focused ultrasound is employed. However, at such low ultrasound frequencies, the focal spot of the ultrasound field is usually larger than 10 mm in length, which is greater than the diameters of most veins. As a result, severe damage can occur to the surrounding tissue and vessel walls. This is especially problematic in areas with delicate structures that have limited surgical options, such as retina vein occlusions where the delicate structure precludes most existing non-pharmacological treatments, and renal vein thrombus where vein access and removal is highly invasive. To increase the efficiency of ultrasound-based thrombolysis, microbubbles can be injected, however, this increases the risk of systemic toxicity and unwanted vascular and organ damages.

Laser thrombolysis is an interventional procedure to re-canalize occluded arteries using light wavelengths that are highly absorbed by the blood clots. Laser light is generally directed to the blood clot through a thin laser fiber. Once laser light is absorbed and the blood clot is heated up, cavitation can occur in the blood clot through vaporization. Then, similar as ultrasound thrombolysis, the expansion and collapse of cavitation can break down the blood clot. Its advantages include low cost, short recovery time and it is generally safe. Laser energy can induce cavitation precisely in blood clots due to the high optical absorption of blood clots and the precision at which the energy can be delivered using the fiber optics. However, the produced cavitation expansion and collapse cannot be well controlled and often are not strong enough. As a result, laser thrombolysis often cannot completely clear thrombotic occlusions in blood vessels, typically leaving residual thrombus on the blood vessel walls, and its efficiency is also questionable in removing residues with high calcium content.

### SUMMARY

In some embodiments, a system for thrombolysis includes an optical energy source, an ultrasound transducer, and an optical conduit for insertion into a vessel. The optical conduit directs optical energy from the optical energy source to a target location at a terminal end of the optical conduit.

In some embodiments, a method of inducing thrombolysis includes positioning an acoustic energy source outside of a patient's body and focused at a target location, positioning an optical conduit inside a vessel of the patient's body with a terminal end of the optical conduit proximate the target location, transmitting an acoustic energy burst to the target location, providing a laser pulse through the optical conduit to the target location, and causing cavitation in the vessel.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims or may be learned by the practice of such embodiments as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1 is a perspective view of a system for inducing thrombolysis in a deep vessels, according to at least one embodiment of the present disclosure;

FIG. 2 is a detail view of a system for inducing thrombolysis in a deep vessels with an intravascular optical conduit, according to at least one embodiment of the present disclosure;

FIG. 3 is a side cross-sectional view of a terminal end of an intravascular optical conduit, according to at least one embodiment of the present disclosure;

FIG. 4A is a side cross-sectional view of a vessel in a tissue with acoustic energy applied alone, according to at least one embodiment of the present disclosure;

FIG. 4B is a side cross-sectional view of a vessel in a tissue with optical energy applied alone, according to at least one embodiment of the present disclosure;

FIG. 4C is a side cross-sectional view of the vessel of FIG. 4A and 4B with optical energy and acoustic energy applied concurrently in an active area, according to at least one embodiment of the present disclosure;

FIG. 5 is a set of charts showing experimentally measured ranges of acoustic pressures that produce thrombolysis with associated optical energy power levels, according to at least some embodiments of the present disclosure; and

FIG. 6 is a set of charts showing experimental thrombolysis treatment times for a matrix of acoustic energies and optical energies, according to at least one embodiment of the present disclosure.

### DETAILED DESCRIPTION

This disclosure generally relates to devices, systems, and methods for removing blood clots. More particularly, the present disclosure relates to combined application of an endovascular laser with ultrasound for thrombolysis for removing blood clots in deep vessels. In some embodiments, systems and methods of photo-mediated ultrasound therapy (PUT) described in the present disclosure may allow blood clot removal with increased precision and/or selectivity when compared to conventional therapies.

PUT includes a combined use of optical energy and acoustic energy to selectively target specific tissue or material in a body. The increased selectivity of PUT compared to conventional therapies may be at least partially related to the endogenous optical contrast between tissue types. Different tissues in an organism contain different concentrations of various chromophores and have different optical absorption spectra. The optical absorption spectra can facilitate the differentiation of tissue types with multi-spectral optical techniques that are highly sensitive and specific. PUT has the unique capability to target, without any exogenous agent, blood clots by taking advantage of the high native contrast in the optical absorption between different other tissues. For example, hemoglobin absorbs more optical energy than other tissues at certain wavelengths, and excitation (and cavitation) can be limited to the blood vessel, providing highly localized treatment. In contrast to conventional therapeutic techniques utilizing ultrasound contrast agents and/or nanoparticles to catalyze cavitation, PUT may produce cavitation in the targeted blood clot or other tissue without additional agents and selectively at the confluence of the applied optical energy and acoustic energy.

The combination of the optical energy and acoustic energy may produce photospallation. Photospallation is the creation of thermoelastic stress in the blood clot via a photoacoustic effect. Upon application of the optical energy, a photoacoustic wave is produced in or on a surface of the blood clot. The oscillating cavitation may then apply mechanical stresses to the blood clot producing thrombolysis.

The PUT techniques described herein combining acoustic energy and optical energy are more precise and require less total energy to be applied to the patient's tissue than conventional acoustic thrombolysis techniques or conventional optical thrombolysis techniques alone. Conventional acoustic thrombolysis uses much higher negative pressures between 14 MPa and 19 MPa (such as described in Maxwell et al., “Noninvasive treatment of deep venous thrombosis using pulsed ultrasound cavitation therapy (histotripsy) in a porcine model”, J Vasc Intery Radiol. 2011 March; 22(3): 369-377. doi:10.1016/j.jvir.2010.10.007) compared to acoustic pressures of less than 5 MPa, as described according to the present disclosure. Conventional optical thrombolysis uses much higher optical energy with greater fluence of 30 to 80 mJ/mm² and pulse lengths of 185 ns (such as described in Nagamine et al., “Comparison of 0.9-mm and 1.4-mm catheters in excimer laser coronary angioplasty for acute myocardial infarction”, Lasers in Medical Science (2019) 34:1747-1754. Doi.org/10.1008/s10103-019-02772) compared to pulse lengths up to 10 ns and fluence up to 500 mJ/cm² (5 mJ/mm²).

Even the combined acoustic energy and optical energy applied by the systems and methods according to the present disclosure is less than the energies applied by the conventional techniques. As described herein, acoustic transducers can have a spot size much larger than the targeted thrombus, resulting in potential damage to the surrounding tissue. Optical conduits increase in diameter according to increases in optical power needed for the application. Therefore, lower optical power allows for smaller diameter conduits that can be more flexible, safer, and able to navigate more vasculature of the patient's body than conduits used for conventional laser thrombolysis. For example, conventional excimer laser catheters used for laser thrombolysis have a diameter of 0.9 mm, 1.4 mm, 1.7 mm, 2.0 mm, while embodiments of laser catheters according to the present disclosure have diameters less than 750 microns (0.75 mm), including a 400-micron (0.40 mm) diameter laser catheter used during the testing described herein.

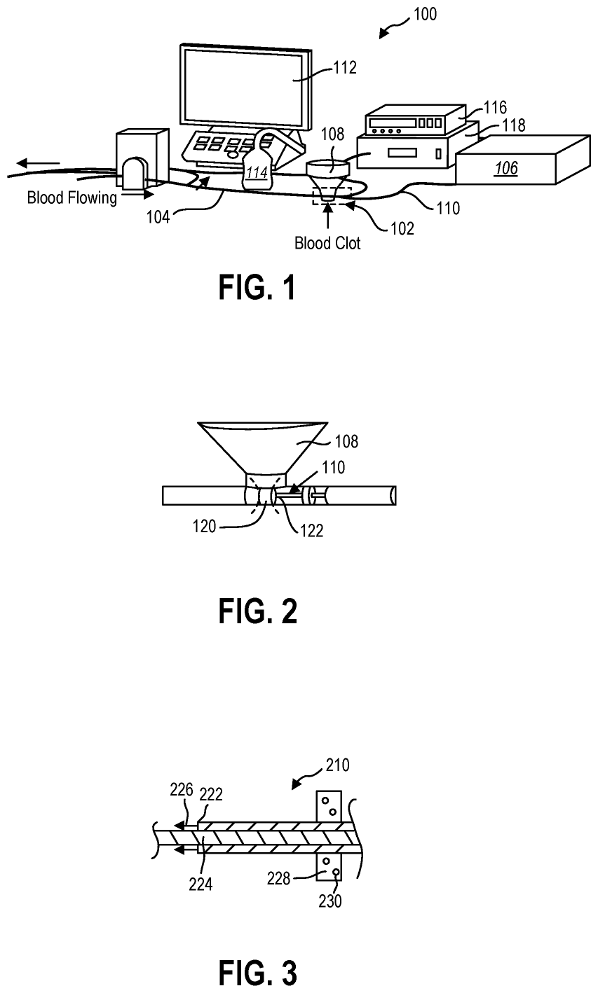

FIG. 1 is a perspective view of an embodiment of a system 100 for producing cavitation in a vessel. FIG. 1 illustrates a bench test system for providing energies to a target location 102 in a simulated blood vessel 104 with a blood clot in the vessel 104. In some embodiments, the system includes an optical energy source 106, such as a laser source, and an acoustic energy source 108, such as a High-intensity focused ultrasound (HIFU) transducer. The laser source is connected to an optical conduit 110, such as an optical fiber, to direct the optical energy to the target location 102. The HIFU transducer is also directed at the target location 102. In some embodiments, neither the acoustic energy nor the optical energy is sufficient to promote cavitation in the vessel 104 individually, allowing each to be used without harming the tissue surrounding the target location 102 when the energies overlap. In some embodiments, the combination of the acoustic energy and the optical energy promotes cavitation through the synchronized application of the energies. For example, the optical energy and acoustic energy are synchronized such that the optical energy creates a bubble, and the acoustic energy expands or collapses the bubble.

In some embodiments, the acoustic energy is able to penetrate through the surrounding tissue to the target location 102 (e.g., the blood clot). The optical energy, however, may have much shorter transmission depths through the tissue, and the optical conduit 110 allows the optical energy to be provided directly to the target location 102 within the vessel 104. The optical conduit 110, therefore, may allow PUT in deep vessels, not otherwise accessible from surface application of optical energy. In some embodiments, the optical conduit 110 is a fiber optic cannula.

The optical energy source 106 and acoustic energy source 108 may be confocal at the target location 102. In some embodiments, the optical energy source 106 and/or acoustic energy source 108 may be operated in a series of pulses. For example, the optical energy source 106 may be pulsed such that at least a portion of the pulse temporally overlaps with a pulse of the acoustic energy source 108. In other examples, the optical energy source 106 may be pulsed during a continuous operation of the acoustic energy source 108. In yet other examples, the optical energy source 106 may be operated continuously while the acoustic energy source 108 is pulsed during the operation of the optical energy source 106.

In some embodiments, the optical energy source 106 and/or the acoustic energy source 108 may be controlled at by a computing device 112. In at least one embodiment, the computing device 112 may include or be in communication with a computer readable medium (CRM) that may contain instructions that, when read by the computing device, cause the computing device to perform one or more methods described herein. For example, the timing of the optical energy source 106 pulse and the acoustic energy source 108 pulse may be coordinated by the computing device 112. In other examples, a series of overlapping pulses from the optical energy source 106 and acoustic energy source 108 may be controlled by the computing device 112. In yet other examples, the computing device 112 may be in communication with one or more sensors 114 configured to detect cavitation or other aspects of the target location 102 and/or tissue 104. In such examples, the computing device 112 may pulse the optical energy source 106 and/or acoustic energy source 108 based at least partially on information communicated by the one or more sensors 114.

In some embodiments, the system 100 may include one or more components to provide additional control over the delivery of acoustic energy. For example, the system 100 may include one or more power amplifiers 116 in communication with the acoustic energy source 108. In examples, the system 100 may include one or more function generators 118 in communication with the acoustic energy source 108 to control the phase and/or frequency of the acoustic energy from the acoustic energy source 108.

In some embodiments, the optical energy source 106 may include one or more lasers. For example, the optical energy source may include an yttrium aluminum garnet (YAG) laser. A neodymium-doped yttrium aluminum garnet (Nd:YAG) pumped optical parametric oscillator (OPO) system may be used as the optical energy source 106 for PUT. The optical energy source 106 may have a peak wavelength in the tuning range. In some embodiments, the peak wavelength may be in a range having an upper value, a lower value, or an upper and lower value including any of 400 nm, 600 nm, 800 nm, 1000 nm, 1200 nm, 1400 nm, 1600 nm, 1800 nm, 2000 nm, 2200 nm, 2400 nm, or any values therebetween. For example, the peak wavelength may be greater than 400 nm. In other examples, the peak wavelength may be less than 2400 nm. In yet other examples the peak wavelength may be in a range of 400 nm to 2400 nm. In further other examples, the peak wavelength may be in a range of 450 nm to 1600 nm. In at least one example, the peak wavelength may be tuned to the peak optical absorption wavelength of the target material, such as hemoglobin.

In some embodiments, an optical power of the optical energy directed at the target location 102 is in a range having an upper value, a lower value, or upper and lower values including any of 1 mW, 2 mW, 4 mW, 6 mW, 8 mW, 10 mW, 15 mW, 20 mW, 40 mW, 60 mW, 80 mW, 100 mW, 200 mW, or any values therebetween. For example, the optical power may be greater than 1 mW. In other examples, the optical power may be less than 200 mW. In yet other examples, the optical power may be between 1 mW and 200 mW. In further examples, the optical power may be between 2 mW and 100 mW. In yet further examples, the optical power may be between 10 mW and 50 mW. In at least one example, the optical power may be 25 mW.

The optical energy source 106 may operate at a repetition rate with a pulse duration. In some embodiments, the optical energy source 106 may be operated at a repetition rate in a range having an upper value, a lower value, or upper and lower values including any of 2 Hz, 4 Hz, 6 Hz, 8 Hz, 10 Hz, 15 Hz, 20 Hz, 40 Hz, or any values therebetween. For example, the repetition rate may be greater than 2 Hz. In other examples, the repetition rate may be less than 40 Hz. In yet other examples, the repetition rate may be in a range of 2 Hz to 40 Hz. In further examples, the repetition rate may be between 6 Hz and 20 Hz. In at least one example, the repetition rate may be 10 Hz.

In some embodiments, the pulse duration may be in a range having an upper value, a lower value, or upper and lower values including any of 0.1 nanosecond (ns), 0.5 ns, 1 ns, 2 ns, 3 ns, 4 ns, 5 ns, 6 ns, 7 ns, 8 ns, 9 ns, 10 ns, or any values therebetween. For example, the pulse duration may be greater than 0.1 ns. In other examples, the pulse duration may be less than 10 ns. In yet other examples, the pulse duration may be between 0.1 ns and 10 ns. In further examples, the pulse duration may be between 2 ns and 6 ns. In at least one example, the pulse duration may be 4 ns.

The optical energy is delivered to the target location 102 and the surface fluence is monitored and controlled during application using the one or more sensors 114 and/or a camera. In some embodiments, the fluence may be in a range having an upper value, a lower value, or upper and lower values including any of 0.1 mJ/cm², 0.5 mJ/cm², 1 mJ/cm², 2 mJ/cm², 4 mJ/cm², 6 mJ/cm², 8 mJ/cm², 10 mJ/cm², 15 mJ/cm², 20 mJ/cm², 40 mJ/cm², 60 mJ/cm², 80 mJ/cm², 100 mJ/cm², 200 mJ/cm², 300 mJ/cm², 400 mJ/cm², 500 mJ/cm², or any values therebetween. For example, the fluence may be greater than 0.1 mJ/cm². In other examples, the fluence may be less than 500 mJ/cm². In yet other examples, the fluence may be between 0.1 mJ/cm² and 200 mJ/cm². In further examples, the fluence may be between 2 mJ/cm² and 100 mJ/cm². In yet further examples, the fluence may be between 3 mJ/cm² and 20 mJ/cm². In at least one example, the fluence may be 4 mJ/cm².

Acoustic energy may be applied to the target location 102 in addition to the optical energy. In some embodiments, an acoustic energy source 108, such as a HIFU transducer or therapeutic ultrasound transducer, may be positioned near or adjacent the target location 102. The acoustic energy source 108 may be used to supply ultrasound bursts to the target location 102. The acoustic energy source 108 may produce an ultrasound pulse through the center of the acoustic energy source 108 that is overlapping the optical energy passing through the optical conduit 110 and directed at the target location 102.

In some embodiments, the acoustic energy source 108 may use an electric potential and/or current to move one or more components of the acoustic energy source 108 and produce an acoustic wave. An annular structure of the acoustic energy source 108 may assist in focusing the acoustic wave through the center of the acoustic energy source 108 and directing the acoustic energy to the target location 102. In some embodiments, the focal length of the acoustic energy source 108 is at least 6 centimeters (cm). In other embodiments, the focal length of the acoustic energy source 108 is at less than 10 cm. The focal length of the acoustic energy source 108 may allow the acoustic energy source 108 to focus the acoustic energy at a target location within the patient's tissue while the acoustic energy source 108 is positioned outside of the patient's body. Application of the optical energy or the acoustic energy individually is insufficient to promote cavitation in microvessels. The concurrent application of the optical energy and the acoustic energy promotes cavitation that promotes thrombolysis of a blood clot.

In some embodiments, a laser pulse is delivered to the target location to overlay the rarefaction phase (maximum negative pressure) at the beginning of each ultrasound burst. Timing of the concurrent energy delivery during rarefaction increases the likelihood of cavitation according to the underlying mechanism. In other embodiments, a laser pulse is delivered to the target location to overlay each rarefaction phase (maximum negative pressure) during each ultrasound burst.

In some embodiments, the therapeutic ultrasound transducer may provide acoustic energy with a maximum positive pressure and/or maximum negative pressure (below atmosphere) in a range having an upper value, a lower value, or upper and lower values including any of 0.1 Megapascals (MPa), 0.2 MPa, 0.3 MPa, 0.4 MPa, 0.5 MPa, 0.6 MPa, 0.8 MPa, 1.0 MPa, 1.2 MPa, 1.4 MPa, 1.6 MPa, 1.8 MPa, 2.0 MPa, 2.5 MPa, 3.0 MPa, 4.0 MPa, 5.0 MPa, or any values therebetween. For example, the pressure maximum may be greater than 0.1 MPa. In other examples, the pressure maximum may be less than 5.0 MPa. In yet other examples, the pressure maximum may be between 0.1 MPa and 5.0 MPa. In further examples, the pressure maximum may be between 0.2 MPa and 2.5 MPa. In yet further examples, the pressure maximum may be between 0.3 MPa and 2.0 MPa. In at least one example, the pressure maximum may be 0.45 MPa.

In some embodiments, the therapeutic ultrasound transducer may provide acoustic energy with a frequency in a range having an upper value, a lower value, or upper and lower values including any of 500 kHz, 550 kHz, 600 kHz, 650 kHz, 700 kHz, 750 kHz, 800 kHz, 850 kHz, 900 kHz, 950 kHz, 1.0 MHz, or any values therebetween. For example, the frequency may be greater than 500 kHz. In other examples, the frequency may be less than 1.0 MHz. In yet other examples, the frequency may be between 500 kHz and 1.0 MHz. In further examples, the frequency may be between 550 kHz and 950 kHz. In yet further examples, the frequency may be between 600 kHz and 900 kHz. In at least one example, the frequency may be 750 kHz.

In some embodiments, the duty cycle of the therapeutic ultrasound transducer may be in a range having an upper value, a lower value, or upper and lower values including any of 0.1%, 1%, 2%, 3%, 4%, 5%, 6%, 7%, 8%, 9%, 10%, or any values therebetween. For example, the duty cycle may be greater than 0.1%. In other examples, the duty cycle may be less than 10%. In yet other examples, the duty cycle may be between 0.1% and 10%. In further examples, the duty cycle may be between 2% and 9%. In yet further examples, the duty cycle of the therapeutic ultrasound transducer may be between 3% and 8% during operation.

In some embodiments, the treatment duration to remove microvessels in the target area may be in a range having an upper value, a lower value, or upper and lower values of 1 second (s), 5 s, 10 s, 15 s, 30 s, 45 s, 60 s, 75 s, 90 s, 105 s, 120 s, or any values therebetween. For example, the treatment duration may be greater than 1 s. In other examples, the treatment duration may be less than 120 s (2 minutes). In yet other examples, the treatment duration may be less than 90 s. In further examples, the treatment duration may be less than 60 s. In at least one example, the treatment duration may be between 15 s and 90 s.

FIG. 2 is a detail side view of the acoustic energy source 108 and the optical conduit 110 directing acoustic energy and optical energy, respectively, at the target location 102. In some embodiments, the target location 102 in within a blood clot 120. In some embodiments, the target location 102 is on a surface of the blood clot 120.

In some embodiments, the optical conduit 110 has a diameter less than 750 microns. In some embodiments, the optical conduit 110 has a diameter less than 500 microns. In at least one embodiment, the optical conduit 110 has a diameter less than 400 microns.

The combined optical and acoustic therapy depends on optical absorption to produce cavitation. In some embodiments, the optical energy has a 532 nm wavelength, at which hemoglobin has strong optical absorptions; hence, a 532 nm wavelength is very effective to produce cavitation and achieve thrombolysis. Longer wavelength light such as 650-nm may also be used to induce cavitation when combined with ultrasound. However, the produced cavitation activity will not be as strong as that at 532 nm because the optical absorption of hemoglobin is relatively weak at 650 nm. As a result, it could take a long period of time or higher ultrasound pressure to achieve effective thrombolysis. In some embodiments, hemoglobin absorption at shorter wavelength such as 400 nm is much stronger than that at 532 nm. Hence, the use of a short wavelength light for thrombolysis may improve the efficiency.

Due to strong optical absorption, short wavelength light (e.g., 400 nm to 500 nm) does not penetrate as deeply into the blood clot 120. Consequently, cavitation can only be induced on a thin surface layer of the blood clot 120, resulting in the removal of the blood clot 120 in a layer-by-layer manner; thereby, the possible damage to the blood vessel wall that is underneath the blood clot 120 may be minimized. The volume of effective cavitation will be reduced using short wavelength light because of less penetration. In some embodiments, the terminal end 122 of the optical conduit 110 includes an optical diffuser tip. A diffuser tip may expand the light illumination area of the optical energy. The diffuser tip may reduce the likelihood of laser-caused charring near the tip, which could otherwise significantly reduce the efficiency of light delivery to the blood clot 120.

FIG. 3 is a side cross-sectional view of an embodiment of an optical conduit 210, according to the present disclosure. In some embodiments, the optical conduit 210 is an optical cannula, which allows for another device or element, such as a guidewire 224, to be positioned in the optical conduit 210. A guidewire 224 may allow precise control of the optical conduit 210 and direction of the optical energy 226 projected by the terminal end 222 of the optical conduit 210.

In some embodiments, the optical conduit 210 configured to be positioned in a blood vessel includes an annular filter 228 positioned circumferentially around the optical conduit 210. The filter 228 may be made of porous materials with pores 230 therethrough. In some embodiments, the pores 230 are less than 100 micrometers (μm). In some embodiments, the pores 230 are less than 50 μm. In some embodiments, the pores 230 are less than 25 μm. When placed in a blood vessel, the filter is squeezed and the sizes of pores 230 are smaller than at full expansion. When the filter is placed downstream, the filter 228 may trap large debris of blood clots that break away during the thrombolysis procedure, further improving the safety potential of combined PUT using intravascular application of the optical energy.

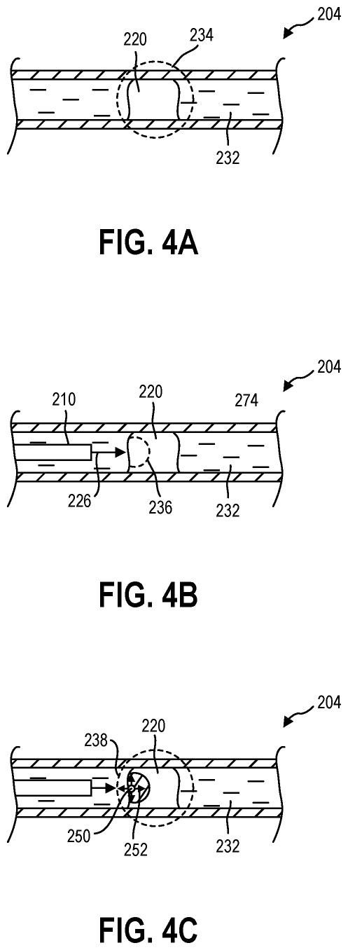

FIG. 4A, 4B, and 4C illustrate the results of an experiment under the described conditions. The photoacoustic signal detected during the experiment indicates the amount of cavitation occurring in the target location and/or microvessel. FIG. 4A is a side cross-sectional view of an embodiment of a vessel 204 in a tissue with acoustic energy applied alone. The vessel 204 contains a fluid 232, such a hemoglobin or other bodily fluid. In some examples, the vessel 204 may be an artery where the fluid 232 is oxygenated hemoglobin. In other examples, the vessel 204 may be a vein where the fluid 232 is deoxygenate hemoglobin. In yet other examples, the vessel 204 may be another vessel of the body containing another fluid 232. An acoustic energy is applied in an acoustic area 234 to the vessel 204 and fluid 232. The acoustic energy alone is insufficient to cause cavitation in the blood clot 220.

FIG. 4B is a side cross-sectional view of the embodiment of a vessel 204 in tissue 204 of FIG. 4A with optical energy applied alone. An optical energy 226 is applied in an optical area 236 to the blood clot 220 via the intravascular optical conduit 210. The optical 226 energy alone is insufficient to cause cavitation in the blood clot 220.

FIG. 4C is a side cross-sectional view of the embodiment of a vessel 204 in FIG. 4A with synchronized optical energy and acoustic energy applied concurrently in an active area 238. For example, the optical energy and acoustic energy are synchronized such that the optical energy creates a bubble, and the acoustic energy expands or collapses the bubble. The synchronized application of the acoustic energy and optical energy produces cavitation in the fluid 232 and/or the blood clot 220. Bubbles 240 forming in the fluid 232 oscillate in diameter, applying a pressure 242 radially outward from the bubbles 240 to the blood clot 220. Cavitation is observed only when ultrasound and laser were applied concurrently, and synchronized application provides the greatest effect.

The safety of thrombolysis treatment with the combined endovascular laser and non-invasive ultrasound is further improved because of the use of the low levels of laser power and ultrasound pressure. The applied laser powers are much lower than that of conventional laser therapies, which are generally above 1 J. The applied ultrasound pressures were also low, resulting in a Mechanical Index (MI) that was much less than 1.9, the safety limit for ultrasound imaging. Consequently, the applied ultrasound pulse will not cause any collateral damage on the vessel wall.

FIG. 5 is a set of charts showing experimentally measured ranges of acoustic pressures that produce thrombolysis with associated optical energy power levels. For example, the application of 50 mW (4 mJ/cm²) of optical power via a laser source and optical conduit in the vessel reduced the needed acoustic pressure from between 1.2 MPa and 1.35 MPa with 0 mW (0 mJ/cm²) down to as low as about 0.5 MPa. An optical power of approximately 25 mW (2 mJ/cm²) and acoustic energy pressure of about 1 MPa, also produced thrombolysis, keeping both the optical energy and the acoustic energy within safe levels that do not harm the surrounding tissue.

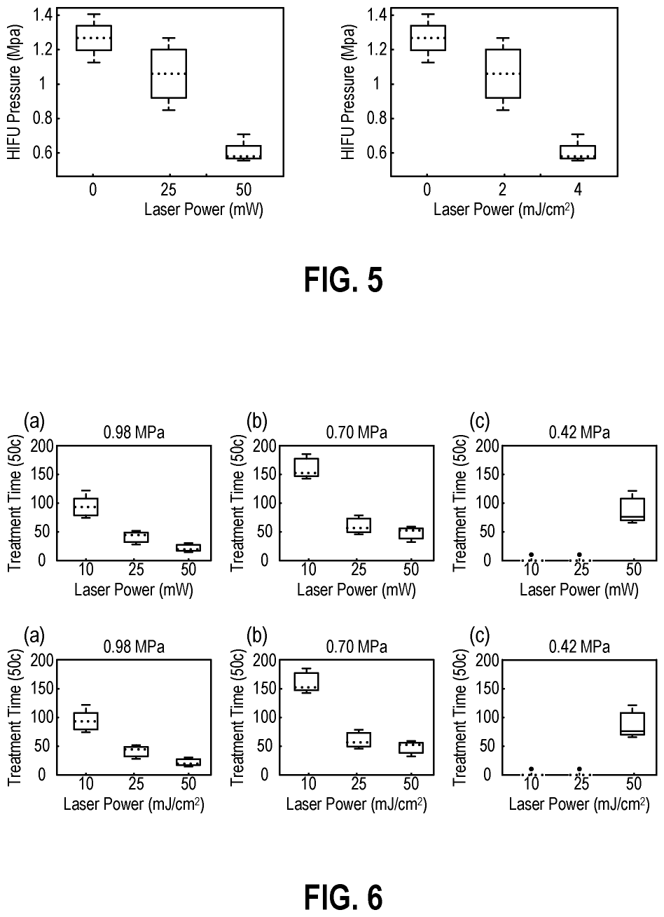

FIG. 6 also illustrates experimental thrombolysis treatment times for a matrix of acoustic energies (e.g., chart (a) being 0.98 MPa, chart (b) being 0.70 MPa, and chart (c) being 0.42 MPa) and optical energies (10 mW, 25 mW, and 50 mW or 0.8 mJ/cm², 2 mJ/cm², 4 mJ/cm²) respectively. In each instance, the total treatment time needed to produce thrombolysis decreased substantially with changes from 10 mW to 25 mW. Smaller improvements were seen from 25 mW to 50 mW, but the treatment time continued to reduce. In the event of 10 mW and 25 mW at 0.42 MPa, no thrombolysis was observed.

There are therapeutic and safety benefits with the combined laser-ultrasound technique described herein. The combination of optical and acoustic energies to achieve thrombolysis is low in risk to the patient by reducing the total energy needed from either of the acoustic or optical energies individually. Direct application of the optical energy from inside the vessel using an intravascular optical conduit allows targeted, low-energy optical energy with an associated filter to safely capture the debris during thrombolysis.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A system for removing blood clots, the system comprising:
an optical energy source;
an ultrasound transducer; and
an optical conduit for insertion into a vessel, wherein the optical conduit directs optical energy from the optical energy source to a target location at a terminal end of the optical conduit.

2. The system of claim 1, where the laser source has a wavelength less than about 800 nm.

3. The system of claim 1, further comprising a particle filter circumferentially attached to the terminal end of the optical conduit.

4. The system of claim 1, the optical energy source being a laser and the optical conduit being a fiber optic.

5. The system of claim 1, the optical conduit having a diameter less than 750 microns.

6. A method for inducing cavitation at a target location, the method comprising:
transmitting an acoustic energy burst into a tissue space; and
providing a sequence of laser pulses through an optical conduit to a target location within the tissue space.

7. The method of claim 6, wherein the target location contains at least a portion of a blood clot.

8. The method of claim 6, wherein the acoustic energy has a pressure of about 0.1 to about 10 MPa.

9. The method of claim 6, wherein the optical conduit is a fiber optic configured for insertion into a vessel.

10. The method of claim 6, wherein an optical power of the laser pulses is between about 1 and 200 mW.

11. The method of claim 6, wherein a laser fluence of the laser pulses is between about 0.1 and 500 mJ/cm².

12. The method of claim 6, wherein a laser power of the laser pulses is less than 100 mW and an ultrasound pressure of the acoustic energy is less than 5 MPa.

13. The method of claim 6, wherein the target region is within a vessel.

14. The method of claim 6, where the vessel is a vein.

15. The method of claim 6, wherein the cavitation induces thrombolysis.

16. The method of claim 6, wherein the acoustic energy burst and the optical energy pulse are synchronized.

17. A method of inducing thrombolysis, the method comprising:
positioning an acoustic energy source outside of a patient's body and focused at a target location;
positioning an optical conduit inside a vessel of the patient's body with a terminal end of the optical conduit proximate the target location;
transmitting an acoustic energy burst to the target location;
providing a laser pulse through the optical conduit to the target location; and
causing cavitation in the vessel.

18. The method of claim 17 further comprising aiming the terminal end of the optical conduit with a guidewire positioned inside the optical conduit.

19. The method of claim 17 further comprising repeating the acoustic energy burst and laser pulse for a treatment time of less than 150 seconds to produce thrombolysis.

20. The method of claim 17 further comprising capturing debris from a blood clot at the target location with a filter positioned on the optical conduit.
